(19) United States
(12) Patent Application Publication
(10) Pub. No.: US 2009/0112299 A1
(43) Pub. Date: Apr. 30, 2009

(54) Direct heating facemask

(76) Inventor: Alfred Chapman, Safety Harbor, FL (US)

Correspondence Address: MCHALE & SLAVIN, P.A., 2855 PGA BLVD, PALM BEACH GARDENS, FL, 33410, US

(21) Appl. No.: 11/932,310

(22) Filed: Oct. 31, 2007

(51) Int. Cl. A61F 7/00

(52) U.S. Cl. 607/109

(57) ABSTRACT

The purpose of this invention is to provide a facemask designed to apply and maintain direct, constant heat in an evenly distributed fashion at a constant temperature of up to about 46° C. to the nasal cavity and surrounding sinuses, thereby creating a thermal environment within the nasal cavity and surrounding area that impedes HRV as well as other heat-labile microorganism replication in order to retard the progression and severity of the common cold and accelerate recovery therefrom. The facemask may include a flexible heating element attached to the inside of the facemask, also an inner lining of foam may be laid over the top of the flexible heating element for added support and insulation. The facemask is secured to the face, e.g. by an elastic strap that wraps around the head and is secured on both ends of the facemask. The heating element is powered by an external power source, e.g. a 9-volt battery, which is attached externally on the elastic strap to avoid interference with the user, and may include electromagnetic shielding to minimize any chance of possible exposure to radiofrequency emissions.

### FIELD OF THE INVENTION

This invention relates generally to a facemask, and more particularly, to a heated facemask for symptomatic relief for the common cold by maintaining heat about the nasal cavity area.

### BACKGROUND OF THE INVENTION

The common cold continues to affect millions worldwide. Although the symptoms last a relatively short period of time, the amount of time lost in the workplace, school or the like are substantial. The common cold is caused by a multiplicity of viral agents, including human rhinovirus and adenovirus. The common cold is most often caused by the human rhinovirus, from herein known as HRV. HRV's replicate optimally at 33° C. or colder, which may partly explain HRV's predilection for the cooler environment of the nasal mucosa, limiting HRV's to upper respiratory infections. In a study conducted by a team of doctors at the Harvard Hospital, Medical Research Council Common Cold Unit, the inhibitory effect of hyperthermia on HRV's was conducted. The inhibitory effect of hyperthermia on HRV replication is temperature dependent, and, under one step multiplication conditions, a 20 min treatment at 45° C. was found to be extremely effective. The most dramatic effect was observed when hyperthermia was applied at 6 hours post infection with a reduction in virus yield of more than 99% relative to that of the control. The study concluded that nasal hyperthermia could improve the course of a common cold and also give immediate relief of symptoms. After 3-7 days, the original viral cause of most colds have lapsed, but complications sometimes manifest themselves. Microorganisms in the respiratory tract can take advantage of the weakened immune system during a common cold and produce co-infections. Middle ear infection (in children) and bacterial sinusitis are common co-infections. A possible explanation for these co-infections is that strong blowing of the nose drives nasal fluids into those areas. Therefore the need to stop a co-infection before it escalates is essential. By not allowing rhinovirus replication in the nasal cavity to overtake the immune system's ability to handle it effectively, the risk for bacterial co-infections and duration of illness is substantially reduced. Rhinovirus replication is heat labile, but whenever heat is used to control microbial growth inevitably both time and temperature are considered. Thus by applying heat at about 45° C. to the nasal cavity and surrounding areas (where viruses tend to replicate during the occurrence of the common cold) rhinovirus replication is inhibited, which helps reduce the amount of recovery time from a cold; as well as prevent co-infections by staving off opportunistic microorganisms that would flourish in an immune compromised scenario.

It is apparent that a facemask device such as to apply direct heat and maintain that heat at a specified temperature in an evenly distributed fashion to the nasal cavity and surrounding sinuses would be highly beneficial in reducing the severity and duration of a common cold. The thermal environment created by such a facemask within the nasal cavity and surrounding area that impedes viral replication would thwart the common cold and help prevent subsequent co-infections.

### DESCRIPTION OF THE PRIOR ART

The prior art discloses numerous facial masks for protecting humans from exposure to various particulate and gaseous matters which incorporate heat sources:

U.S. Pat. No. 6,974,470 B2, to Tsunakawa et al. discloses a heat generative, steam generative facial sheet that is planar and is adapted to cover a part or the whole of a wearer's face in intimate contact. The planar heat generator is to generate heat upon contact with oxygen and thereby to generate steam. The planar heat generator contains a metal, activated carbon, a fibrous material an electrolyte, and water. It is capable of raising the surface temperature of the facial skin to a desired level in a short time; a wearer can enjoy facial steaming without wait. Since steam generation last long enough to open the pores sufficiently, the facemask is useful for facial cleansing and makeup removal.

U.S. Pat. No. 4,793,343, to Cummins et al. discloses a facemask for use in cold weather to supply warm air for inhalation. The facemask has inlet and outlet check valves and a cold air intake chamber with an electric heater element therein, which heats the cold air to supply warm air for inhalation. The heater element maintains the heated air in a predetermined temperature range and is powered by a portable battery pack.

U.S. Pat. No. 4,620,537, to Brown discloses a breathing mask which has the intake air heater and heat and moisture exchange media located below the nose and situated for minimum exposure to heat transfer to the exterior. The mounting of thereof is somewhat cantilevered from the mask, to minimize direct heat transfer to the exterior. Electric heating element and energy pack are mounted in the mask itself at a relatively low level. The incoming air flows primarily through a main passageway from the heating element means to a heat and moisture exchanging and filter medium and from there to the mouth and/or nose of the individual wearing the mask.

U.S. Pat. No. 3,249,108, to Terman discloses a mask for protecting the respiratory tract pf the wearer from cold air or wind. The mask is made of any suitable air pervious material; the mask contains a pocket for holding a removable heating element. The shape of the mask forms an air pocket on front of the oral orifice and jest below the nasal orifices of the wearer. The heating element comprises of a few turns of wire adapted to be heated by passage of an electrical current therethrough. The wire is encapsulated in a suitable insulator. A battery operated electrical heating element is preferred to maintain a uniform temperature within a desired range. The wires of the heating element are connected to a plug that is adapted to be connected to a battery carried in any convenient place such as, a jacket, pocket, or purse of the wearer. When the mask is being worn and heating element is engaged it insures that all the air entering the respiratory tract of the wearer is warmed. Part of the air passes through the apertures in the heating element, and the rest of the air passes close enough to the heating element to be warmed thereby.

U.S. Pat. No. 5,511,541, to Dearstine discloses a warm air mask comprising of a facial covering with three oval inhale ports formed in the covering and extending therethrough, a single exhale port formed centrally in the covering and extending therethrough, an array of electrical resistance wires for generating heat when electrically activated, the wires of the array being positioned in each inhale port with an associated electrical power source and a switch secured adjacent to the covering for the selectively activating and inactivating the resistance wires.

These prior art patents fail to teach or suggest, either alone or in combination, a facemask for maintaining an elevated temperature effective to retard or substantially eliminate replication of microorganisms associated with symptoms of the common cold when placed in juxtaposed relation to the face of a user, which comprises, in combination, a facial mask backbone, having an upper edge positionable above a user's eyes, a lower edge positionable below a user's nose, and spaced apart side edges positionable over a user's temple area, a topside, a backside, and a plurality of ports for overlying the user's facial cavities; a heating element, in juxtaposed relation with the facial mask backbone, the heating element being electronically coupled to a power supply, for producing heat in an amount effective to maintain the elevated temperature; a securement strap, in removable engagement with the facial mask backbone on the side edges for securement of the facial mask backbone to the user's face; and a power supply, in removable engagement with the securement strap and electronically coupled to the heating element; wherein the electronic coupling of the power supply and heating element provides sufficient heat to maintain a temperature within the nasal cavity and surrounding area effective to retard or substantially eliminate growth of heat labile viruses and microorganisms within the nasal cavity and surrounding area associated with occurrence of the common cold.

### SUMMARY OF THE INVENTION

As a consequence of these aforementioned problems, it is an objective of the present invention to provide a facemask designed to apply and maintain heat in an evenly distributed fashion at a constant temperature of approximately 45° C. to the nasal cavity and surrounding sinuses, thereby creating a thermal environment within the nasal cavity and surrounding area that impedes HRV replication in order to prevent, forestall, and help facilitate recovery from the common cold and the co-infections associated therewith.

Accordingly, it is an objective of the present invention to provide relief from the common cold by impeding the replication of HRV, thermal-sensitive Adenovirus strains and any other cold-causing heat-sensitive viral strains.

It is a further objective of the present invention to provide a facemask that is durable and can be reused multiple times.

It is another objective of the present invention to provide a facemask that can be comfortably worn at all times, specifically at bedtime.

It is an additional objective of the present invention to provide a facemask molded from hard ABS (ABSYLUX) to fit the contours of an average human face.

It is another additional objective of the present invention to provide a facemask base whereby all the other elements of the present invention are attached to said facial base.

It is another objective of the present invention to provide a facemask having openings at the nasal cavity and the eyes.

It is still another objective of the present invention to provide a flexible heating element, for example, etched foil.

It is yet another objective of the present invention to provide a heating capable of supplying and maintaining a temperature to a predefined area in an evenly distributed fashion.

It is a further objective of the present invention to provide a heating element capable of reaching a temperature within the range of 40°-46.11° C.

It is still a further objective of the present invention to provide a heating element that is thermostatically controlled to maintain temperature at or near a desired set point.

It is hitherto another objective of the present invention to provide a foam padding to provide comfort, support, and insulation. The foam padding is preferably high-density memory foam used for comfort on the face. The foam padding is positioned on the inside of the facemask and atop of the heating element to provide even heat distribution and insulation.

It is yet still another objective of the present invention to provide a securement strap to hold the facemask in place.

It is yet still another additional objective of the present invention to provide a portable power supply, for example a 9-volt disposable dry cell.

It is another objective of the present invention to provide a power supply secured to the securement strap.

It is a further objective of the present invention to provide electromagnetic field shielding, for example made of a cobalt based alloy that wraps around the power supply to protect from EMF radiation that the power supply may emit.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of the direct heating facemask in accordance with one embodiment of the present invention.

FIG. 2 is a back view of the direct heating facemask in accordance with one embodiment of the present invention.

FIG. 3 is a side view of the direct heating facemask in accordance with one embodiment of the present invention.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

The present invention is directed toward a facemask for maintaining an elevated temperature effective to retard or substantially eliminate replication of microorganisms associated with symptoms of the common cold when placed in juxtaposed relation to the face of a user. The facemask includes a facial mask backbone, having an upper edge positionable above a user's eyes, a lower edge positionable below a user's nose, and spaced apart side edges positionable over a user's temple area, a topside, a backside, and a plurality of ports for overlying said user's facial cavities. The facemask further includes a heating element, in juxtaposed relation with said facial mask backbone, said heating element being electronically coupled to a power supply, for producing heat in an amount effective to maintain said elevated temperature. Additionally included are a securement strap, in removable engagement with said facial mask backbone on said side edges for securement of said facial mask backbone to said user's face; and a power supply, in removable engagement with said securement strap and electronically coupled to said heating element. In use, the electronic coupling of said power supply and said heating element provides sufficient heat to maintain a temperature within said nasal cavity and surrounding area effective to retard or substantially eliminate growth of heat labile viruses and microorganisms within said nasal cavity and surrounding area associated with occurrence of the common cold.

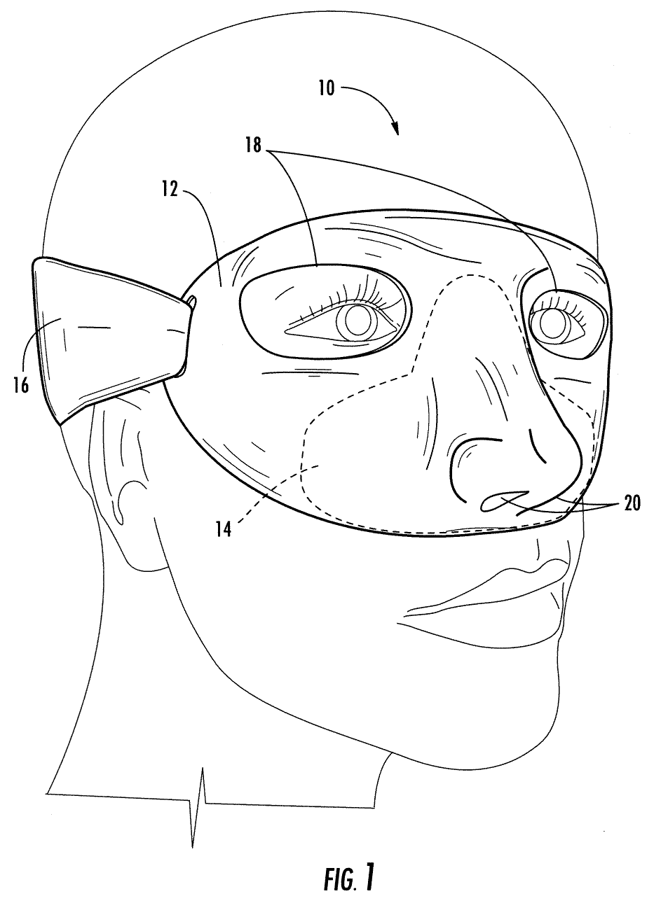

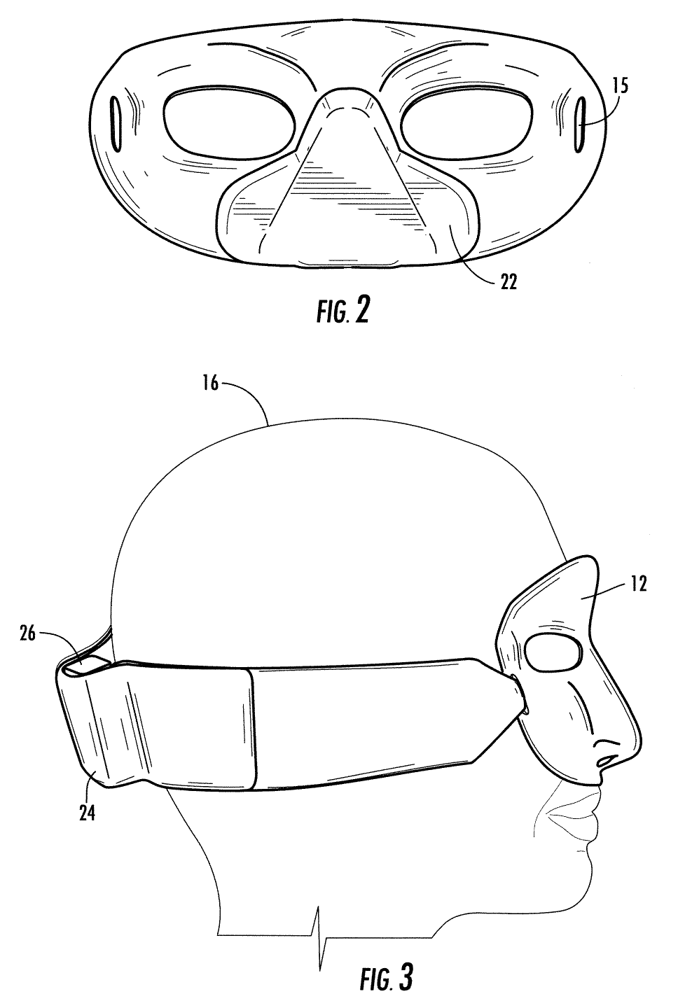

Referring now to FIGS. 1-3, wherein like elements are numbered consistently throughout, FIG. 1 illustrates one embodiment of the direct heating facemask according to the present invention, generally referenced herein as 10. The direct heating facemask 10 begins with a base, herein known as the facial mask backbone 12. The facial mask backbone 12 is specifically contoured to fit the face of an average human. The facial mask backbone 12 is heat vacuumed molded to adapt to the different contours of an average human face. In an illustrative, albeit non-limiting embodiment, the facial mask backbone 12 is made from FDA approved food packaging-grade acrylonitrile-butadiene-styrene, e.g. ABSYLUX, however it is contemplated that the facial mask backbone 12 may be constructed of other similarly performing materials. In a preferred embodiment, the facial mask backbone 12 has a top edge that is positioned above the eyes of a user, a bottom edge that is positioned just below the nose of a user, and side edges that are positioned at or near the temple of a user, alternative embodiments may be constructed to cover only near the nasal cavity area, or extend as far up as the hair, or extend as far down as the mouth. On the facial mask backbone 12 a plurality of ports or openings are formed, specifically, the eye ports 18 and nasal ports 20. The eye ports 18 are an opening on the facial mask backbone 12 that allows a user to see properly when the direct heating facemask 10 is being worn. The nasal ports 20 allow a user to breathe normally through the nostrils through the openings on the nasal ports 20.

On each of the side edges of the facial mask backbone there lays an opening called the attachment site 15. At the attachment site 15 a securement strap 16 is attached to the facial mask backbone 12. The securement strap 16 is preferably made from a one piece elastic band, that extends from one side edge of the facial mask backbone 12 to the opposite side edge of the facial mask backbone 12. The securement strap 16 is flexible enough to expand and contract for varying head sizes. It is contemplated that the securement strap 16 include a buckle, belt, Velcro, or the like. The securement strap 16 is to provide longevity and comfort, as the direct heating facemask 10 will be re-used multiple times and will be placed on the head of a user for an extended period of time. Comfort is essential, as the direct heating facemask 10 will be used at all times of the day, specifically at bedtime as it is not advantageous to have a direct heating facemask 10 that obstructs regular sleeping habits.

With reference to FIGS. 1-3, on the inside of the facial mask backbone 12 a heating element 22 is attached by an FDA-approved, human-safe non-heat sensitive adhesive. The heating element 22 is illustrated as comprising flexibility, such as etched foil, and measuring approximately 0.05 mm thick. The heating element 22 is custom designed to fit into compound curves within the facial mask backbone 12. The heating element 22 is placed to specifically surround the nasal cavity and surround areas of a user. The flexible heating element is thermostatically controlled and capable of reaching temperatures within the range of 40° C. and 46° C. The heating element 22 is electronically coupled to a power supply 24, located on the securement strap 16. It is contemplated that heating element 22 are comprised of other similar direct heating methods such as PTC ceramic materials. The power supply 24 is preferably a 9-volt dry cell disposable battery, however alternatives in the form of 12-volt, 18-volt, solar power, etc. are contemplated. In a particular embodiment, the power supply 24 is enclosed by a battery shield 26, which is made of a cobalt-based alloy, to provide protection from any electromagnetic radiation that the power supply 24 may give off. It is preferred that the battery shield 26 and power supply 24 are attached to the securement strap 16 to provide freedom to a user so as not having to hold, clip, or pocket the power supply 24 during usage. However, in the alternative the power supply 24 and battery shield 26 may be attached externally.

On the inside of the facial mask backbone 12 a heating element 22 is specially contoured to fit curves on the facial mask backbone 12; Atop of the heating element 22 and the surrounding facial mask backbone 12 is an insulation material 14. The insulation material 14 is constructed of foam padding, e.g. a memory-foam type padding, and in a particular embodiment, a 5 mm pad of S-90R memory foam. The insulation material 14 is attached to the inside of the facial mask backbone 12 using FDA-approved, human-safe adhesive and a pneumatic press. The insulation material 14 provides comfort to the user, because it adapts to the special contours of each individual face. The insulation material 14 also serves as a conductor for heat that is given off from the heating element 22, thus providing a heat transfer material. Because the heating element 22 is capable of reaching temperatures of up about 46° C., the foam padding provides a layer of insulation from the heating element 22 thus lowering the temperature directly applied to the nasal cavity and surrounding area by about 1° C., as well providing even heat distribution.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A facemask for maintaining an elevated temperature effective to retard or substantially eliminate replication of microorganisms associated with symptoms of the common cold when placed in juxtaposed relation to the face of a user comprising:
a facial mask backbone, having an upper edge positionable above a user's eyes, a lower edge positionable below a user's nose, and spaced apart side edges positionable over a user's temple area, a topside, a backside, and a plurality of ports for overlying said user's facial cavities;
a heating element, in juxtaposed relation with said facial mask backbone, said heating element being electronically coupled to a power supply, for producing heat in an amount effective to maintain said elevated temperature;
a securement strap, in removable engagement with said facial mask backbone on said side edges for securement of said facial mask backbone to said user's face; and
a power supply, in removable engagement with said securement strap and electronically coupled to said heating element;
wherein said electronic coupling of said power supply and said heating element provides sufficient heat to maintain a temperature within said nasal cavity and surrounding area effective to retard or substantially eliminate growth of heat labile viruses and microorganisms within said nasal cavity and surrounding area associated with occurrence of the common cold.

2. The facemask of claim 1, capable of maintaining a temperature within the range of about 40° C. to about 46° C. within the nasal cavity and surrounding area in juxtaposed relation with said facemask, capable of maintaining heat juxtaposed to the nasal cavity and surrounding area at a temperature between 40° C. to 46° C.

3. The facemask of claim 1, wherein said heating element is flexible.

4. The facemask of claim 3 wherein said flexible heating element is an etched foil heater.

5. The facemask of claim 1, further including an insulated element secured to said backside of said facial mask backbone.

6. The facemask of claim 5, wherein said insulation is a memory foam pad.

7. The facemask of claim 1, wherein said power supply is a battery.

8. The facemask of claim 7, wherein said battery is attached to said securement strap.

9. The facemask of claim 8, wherein said attachment includes a battery shield effective to protect the user from electromagnetic field radiation.

10. The facemask of claim 1, wherein said facial mask backbone is molded from an acrylonitrile-butadiene-styrene.

11. The facemask of claim 1, wherein said securement strap is an elastic band adapted for conforming around a human head.

12. The facemask of claim 1, wherein said user's facial cavities include nostrils and eyes.

13. The facemask of claim 1, wherein said heating element provides constant thermostatically regulated temperature.
